(19) United States
(12) Patent Application Publication
(10) Pub. No.: US 2024/0017396 A1
(43) Pub. Date: Jan. 18, 2024

(54) TORSO SUPPORT AND A WEARABLE SUPPORT SYSTEM COMPRISING THE TORSO SUPPORT

(71) Applicant: Laevo B.V., Rijswijk (NL)

(72) Inventors: Boudewijn Martin WISSE, Rijswijk (NL); Sebastiaan Johannes WAGEMAKER, Rijswijk (NL); Jaromir VAN DE GUCHTE, Rijswijk (NL); Michael Martinus HÖLSCHER, Rijswijk (NL)

(21) Appl. No.: 18/033,424

(22) PCT Filed: Oct. 18, 2021
(86) PCT No.: PCT/NL2021/050627

§ 371 Date: Apr. 24, 2023

(30) Foreign Application Priority Data
Oct. 30, 2020 (NL) 2026803

(51) Int. Cl. B25J 9/00

(52) U.S. Cl. CPC B25J 9/0006

(57) ABSTRACT

A torso support for bearing upon a chest and/or a back of a human body includes a frame, and first and second connection arrangements. The frame includes a resilient stay arranged to extend substantially alongside the back of the human body and arranged for being in a first state when being unloaded, and arranged to be biased back to the first state when being into a second state corresponding with a leaning or bending position of the human body. The first connection arrangement arranged for connecting the torso support to the human body. The second connection arrangement arranged for connecting the torso support to a wearable support structure for at least further partly supporting the human body during leaning or bending over. The wearable support system includes the torso support and the wearable support structure.

According to a first aspect, the present disclosure relates to a torso support for bearing upon a chest and/or a back of a human body and arranged for at least partly supporting said human body during leaning or bending over.

According to a second aspect, the present disclosure relates to a wearable support system comprising a torso support according to the first aspect of the present disclosure and a wearable support structure for at least further partly supporting said human body during leaning or bending over.

The torso support according to the present disclosure comprises:
a frame comprising a resilient stay arranged to extend substantially alongside said back of said human body and arranged for being in a first state when being unloaded, and arranged to be biased back to said first state when being into a second state corresponding with a leaning or bending position of said human body;
a first connection arrangement arranged for connecting said torso support to said human body, wherein said first connection arrangement is connected to said frame at a first location that, in use, is provided at an upper part of said back of said human body, and a second location that, in use, is provided at a lower part of said back of said human body.

By providing the torso support with the frame and the first connection arrangement, support of the human body during leaning and bending over may be provided without the need for wearing an exoskeleton such as for example disclosed in NL2014451. The present disclosure relies at least partly on the insight that by providing a torso support according to the present disclosure a human body may be supported during a leaning or bending position in a relative practical manner without the need for a relative complex and large structure. The frame of the torso support according to the present disclosure is maintained in a position relative to the back of the human body during leaning and bending over such that the frame may support the back of the human body in a relative comfortable manner.

Within the context of the present disclosure, the chest and the back of the human body, referred to in relation to the torso support being arranged for bearing upon the chest and/or the back of said human body, are to be understood as a part of the human body between the shoulders and the lumbar area of the human body. In other words, when a user is wearing the torso support the torso support, or at least the frame thereof, bears upon a part of the human body that is between the shoulders and the lumbar area of the user.

Within the context of the present disclosure the upper part of the back of the human body may be understood as a part of the human body at a location of the human body corresponding to the cervical spine or the vertebrae C4-C6 and the upper part of the thoracic spine or the vertebrae Th1-Th3 of said back of said human body

Within the context of the present disclosure the lower part of the back of the human body may be understood as a part of the human body at a location of the human body corresponding to a lower part of the thoracic spine or vertebrae Th9-Th12 of said back of said human body.

The torso support may further comprise a second connection arrangement arranged for connecting said torso support to a support structure, preferably a wearable support structure, for at least further partly supporting said human body during leaning or bending over.

Providing the second connection arrangement is beneficial for allowing a wearable support structure to be coupled to the torso support in a relative practical manner for further relieving the human body during leaning or bending over. By coupling the torso support to a known wearable support structure, the torso support and the wearable support structure may together support said human body thereby realising a relative comfortable experience to the user.

In this regard, it is noted that NL2014451 discloses a wearable support structure comprising a chest support arranged for bearing upon a chest of said human body. Although this chest support functions well, the coupling, in use, of the wearable support structure with the human body above the hips of the human body may be improved to realise a more comfortable experience for the user.

The torso support according to the present disclosure allows for an improved coupling, in use, of the wearable support structure with the human body above the hips of the human body to realise a more comfortable experience for the user while providing a relative high level of support to the human body.

Preferably, said wearable support structure comprises a third connection arrangement arranged for connecting said wearable support structure to said torso support, wherein said wearable support structure is connectable to said second connection arrangement via said third connection arrangement.

It is advantageous, if said wearable support structure comprises an elongated resilient stay, preferably two elongated resilient stays, connected to said third connection arrangement, arranged to extend substantially alongside said human body and arranged for being in a first state when being unloaded, and arranged to be biased back to said first state when being into a second state corresponding with a leaning or bending position of said human body.

Preferably, said wearable support structure comprises:
two thigh supports, each arranged for bearing upon a respective thigh area of said human body, arranged for being in a first state when being unloaded, and arranged to be biased back to said first state when being into a second state corresponding with a leaning or bending position of said human body; and/or
two pivoting arrangements, each connecting said elongated resilient stay, preferably each connecting a respective elongated resilient stay of said two elongated resilient stays, to a respective thigh support of said two thigh supports and allowing said respective elongated resilient stay and said respective thigh support to pivot about a main axis.

The second connection arrangement is preferably arranged for allowing a force, in a direction away from the human body, to be applied to the torso support, via the second connection arrangement. This is beneficial for allowing a wearable support structure to further relief the human body by applying a force, via the second connection arrangement, to the human body.

Preferably, said first connection arrangement is arranged to be worn, by said human body, in a tight-fitting manner such that said resilient stay is maintained in said second state during leaning or bending position of said human body.

It is beneficial, if said frame is arranged such that, in use, a shape of said resilient stay, between said first location and said second location, corresponds to a shape of said back of said human body. In other words, it is beneficial if a shape of the resilient stay follows the shape of the back of the human body. This is beneficial for realising a relative comfortable experience for the user wearing the torso support.

In this regard, it is beneficial if said frame is provided such that, in use, said resilient stay, between said first location and said second location, is at a distance from said back of said human body. This is beneficial for avoiding, or at least significantly reducing the risk, that the resilient stay between the first and second location presses against the back of the human body thereby realising a relative comfortable experience for the user.

In an embodiment of the torso support according to the present disclosure, said frame, preferably said resilient stay, is provided with a resilient padding material for maintaining said resilient stay at a distance of said back of said human body. This is beneficial for avoiding, or at least significantly reducing the risk, that a relative large pressure is applied, by the resilient stay, between the first and second location against the back of the human body.

Preferably, a spring rate of said padding material is lower than a spring rate of said resilient stay.

Preferably, said second connection arrangement is connected to said frame between said first location and said second location. This allows for applying a force in a direction away from the human body, by the wearable support structure, to the torso support, via the second connection arrangement. This is beneficial for realising a relative large force, by the wearable support system, to the torso of the user during leaning or bending over, thereby realising a relative comfortable experience to the user.

In a practical embodiment of the torso support according to the present disclosure, said second connection arrangement is moveable along said frame between said first location and said second location. This is beneficial for realising a relative comfortable experience for users of different height.

Moreover, the second connection arrangement being moveable between the first location and the second location is beneficial for allowing a user to adapt the distribution of the force applied to the human body, via the torso support. By positioning the second connection arrangement in a predetermined position the distribution of the support provided to the human body may be set according to a preference of the user. As an example, by placing the second connection arrangement closer to the first location than to the second location, the force applied to the human body, via the torso support, will result in more support to the upper part of the back of the user than to the lower part of the back of the user. Alternatively, by placing the second connection arrangement closer to the second location than to the first location, the force applied to the human body, via the torso support, will result in more support to the lower part of the back of the user than to the upper part of the back of the user.

In this regard, it is beneficial if said torso support is arranged for fixating said second connection arrangement at a plurality of positions between said first location and said second location along said frame to said frame. This is beneficial for allowing a user to adapt the torso support in a relative durable manner for reproducing a relative comfortable experience at relative little effort each time the user wears the torso support.

Preferably, said frame is detachably connected to said first connection arrangement. This is beneficial for allowing a user to adapt the level of support to the human body and/or adapt the torso support the a height of the user for instance by exchanging the frame with another frame.

In a very practical embodiment of the torso support according to the present disclosure, said first connection arrangement comprises a textile arranged for bearing upon said chest of said human body.

Preferably, said first connection arrangement is formed as a vest. This is beneficial for realising a relative comfortable experience for the user wearing the torso support.

The vest may further be beneficial for allowing a user to attach items such as a mobile electric device or other attribute to the vest. For this purpose, it is beneficial if the vest is provided with an attachment and/or fastening arrangement for allowing the user to attached and/or fasten a mobile electronic user device, such as a cell phone or a scanner to the vest.

In this regard, it is advantageous if said vest comprises two vest parts that are mutually connectable via a connection arrangement such as a zipper or buttons.

In this regard, it is beneficial if said first connection arrangement comprises a strap for adjusting the shape of the vest. This is beneficial for allowing a user to adapt the vest to the shape of the human body thereby allowing said torso support to be worn in in a relative practical manner a tight-fitting manner such that said resilient stay is maintained in said second state during leaning or bending position of said human body.

Preferably, each vest part of said two vest parts is provided with said strap, wherein said strap is arranged to adhere to a surface of said vest part for allowing said user to adapt a fitting of said torso support.

Preferably, said strap and said vest parts are provided with hook and loop fasteners.

Preferably, said further resilient stay is provided with a through hole, wherein said strap extends through said through hole for attaching said strap to said frame.

It is advantageous if said frame comprises a further stay, preferably a further resilient stay, perpendicular to said resilient stay such that, in use, said further stay extends along a width of said back of said human body. Providing the further stay is beneficial for avoiding, or at least significantly reducing, squeezing of the human body near the further resilient stay.

In an embodiment of the torso support according to the present disclosure, said frame comprises a yet further stay, preferably a yet further resilient stay, wherein said resilient stay and said yet further stay are provided such that said frame is X-shaped, preferably, wherein said second connection arrangement is provided at a crossing of said resilient stay and said yet further stay. Providing an X-shaped frame is beneficial for realising a relative comfortable experience while providing a relative large stability to the human body.

Preferably, said torso support comprises a detector arrangement arranged for detecting a physical state of said human body and/or for detecting forces and deflections of said frame.

Within the context of the present disclosure the physical state of the user is to be understood as a posture of the human body by detecting an orientation of the torso support and/or the frame. In addition, or alternatively, the physical state may comprise an effort exerted by the human body that may by detected for instance by detecting a temperature, temperature or sweat level of the human body.

In an embodiment of the torso support according to the present disclosure, said frame comprises a neck support for support a neck of said human body.

Preferably, said frame is arranged for connecting to a hip part of said wearable support structure for limiting rotation of said hip part during donning and doffing of said wearable support structure.

According to the second aspect, the present disclosure relates to a wearable support system comprising a torso support according to the first aspect of the present disclosure and a wearable support structure for at least further partly supporting said human body during leaning or bending over, wherein said wearable support structure comprises a third connection arrangement arranged for connecting said wearable support structure to said torso support, wherein said wearable support structure is connected to said second connection arrangement via said third connection arrangement.

Embodiments of the wearable support system correspond to embodiments of the torso support according to the first aspect of the present disclosure. The advantages of the wearable support system correspond to the advantages of the torso support according to the first aspect of the present disclosure presented previously.

Preferably, said second connection arrangement is rotatably connected to said third connection arrangement. This is beneficial for providing the user a relative comfortable experience to the user.

In this regard, it is advantageous if said second connection arrangement is rotatably connected to said third connection arrangement such that said second connection arrangement is rotatable relative to said third connecting arrangement over a predetermined angle for limiting rotation of said torso support relative to said wearable support structure. This is beneficial for providing the user a relative comfortable experience to the user.

In this regard, it is advantageous if said second connection arrangement is rotatably connected to said third connection arrangement such that said second connection arrangement is rotatable relative to said third connecting arrangement over a predetermined angle for limiting rotation of said torso support relative to said wearable support structure preferably during donning and doffing of said wearable support system. This is beneficial for providing the user a relative comfortable experience to the user.

Preferably, said predetermined angle is in the range of 45-135 degrees, more preferably 45-90 degrees. This is beneficial for providing the user a relative comfortable experience to the user while avoiding a system that is relatively difficult to handle during donning and doffing. In addition, the predetermined angle may be beneficial for avoid, or reduce the risk of injuring the human body due to a to large angle.

Preferably, said wearable support structure is arranged for bearing against a lumbar area and/or a thigh area of said human body. This is beneficial for providing a relative comfortable experience to the user during leaning or bending over.

In a practical embodiment of the wearable support system according to the present disclosure, said wearable support system is arranged for bearing against said lumbar area and/or said thigh area of said human body only via said wearable support structure.

Preferably, said wearable support structure is arranged for bearing upon said chest and/or said back of said human body via said torso support, more preferably said wearable support structure is arranged for only bearing upon said human body via said chest and/or said back of said human body via said torso support.

Preferably, said wearable support structure comprises an elongated resilient stay, preferably two elongated resilient stays, connected to said third connection arrangement, arranged to extend substantially alongside said human body and arranged for being in a first state when being unloaded, and arranged to be biased back to said first state when being into a second state corresponding with a leaning or bending position of said human body.

In an embodiment of the wearable support system according to the present disclosure, the wearable support structure comprises:
two thigh supports, each arranged for bearing upon a respective thigh area of said human body, arranged for being in a first state when being unloaded, and arranged to be biased back to said first state when being into a second state corresponding with a leaning or bending position of said human body;
two pivoting arrangements, each connecting said elongated resilient stay, preferably each connecting a respective elongated resilient stay of said two elongated resilient stays, to a respective thigh support of said two thigh supports and allowing said respective elongated resilient stay and said respective thigh support to pivot about a main axis.

In this regard, it is beneficial if said wearable support structure comprises a movement arrangement arranged for maintaining said elongated resilient stay, preferably said two elongated resilient stays, and said two thigh supports in said first states when said human body is in an upright position and arranged for maintaining a pivot angle about said main axis of said respective elongated resilient stay of said elongated resilient stay, preferably said two elongated resilient stays, substantially equal to a pivot angle about said main axis of said respective thigh support of said two thigh supports when said human body is in said upright position.

By providing the movement arrangement it is avoided that the two elongated resilient stays of said wearable support structure are in said second state when said human body is in an upright position. The movement arrangement realises that the elongated resilient stay, preferably the two elongated resilient stays, are in said second position only when said human body is leaning or bending. This allows the user to move, for instance during walking, in the upright position without experiencing a biasing force, or at least experiencing only a relative small biasing force from the wearable support structure.

An additional advantage of providing the movement arrangement is that there is no need for a user of the wearable support structure according to the present disclosure to actively bring the wearable support structure in a condition wherein the user may move freely without experiencing the biasing force.

The movement arrangement accommodates for out-of-phase movements of the user of the wearable support structure in the sagittal plane of the human body.

The torso support and the wearable support system according to the present disclosure will now be explained by means of a description of preferred embodiments of a torso supports according to the present disclosure and a wearable support system according to the present disclosure, in which reference is made to the following schematic figures, in which:

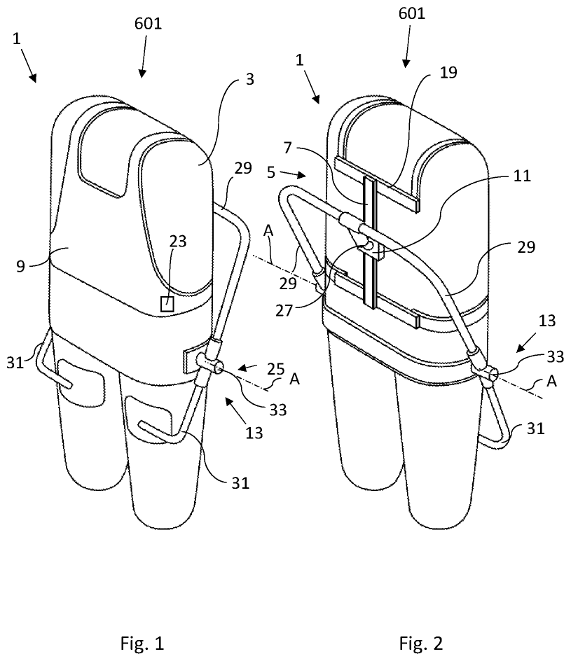

FIGS. 1 and 2: an isometric view of a human body wearing a wearable support system according to the present disclosure is shown;

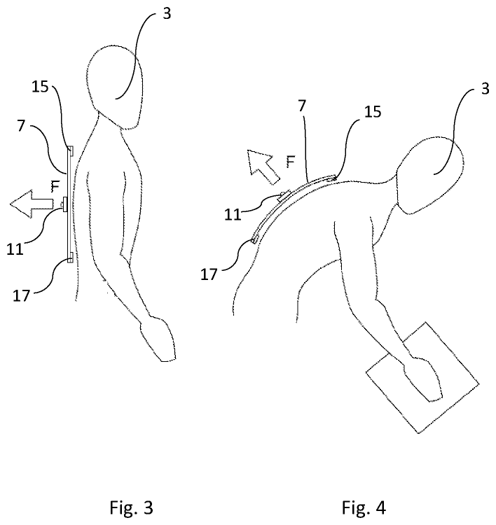

FIGS. 3 and 4: a side view of a human body and elements of a torso support according to the present disclosure is shown;

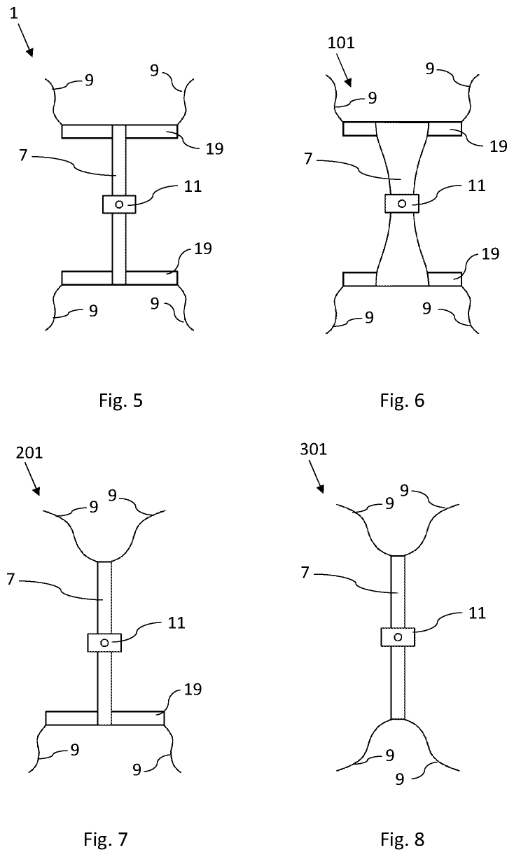

FIG. 5-10: torso supports according to the present disclosure are shown;

FIGS. 11 and 12: a detail of the torso support from FIG. 10 is shown;

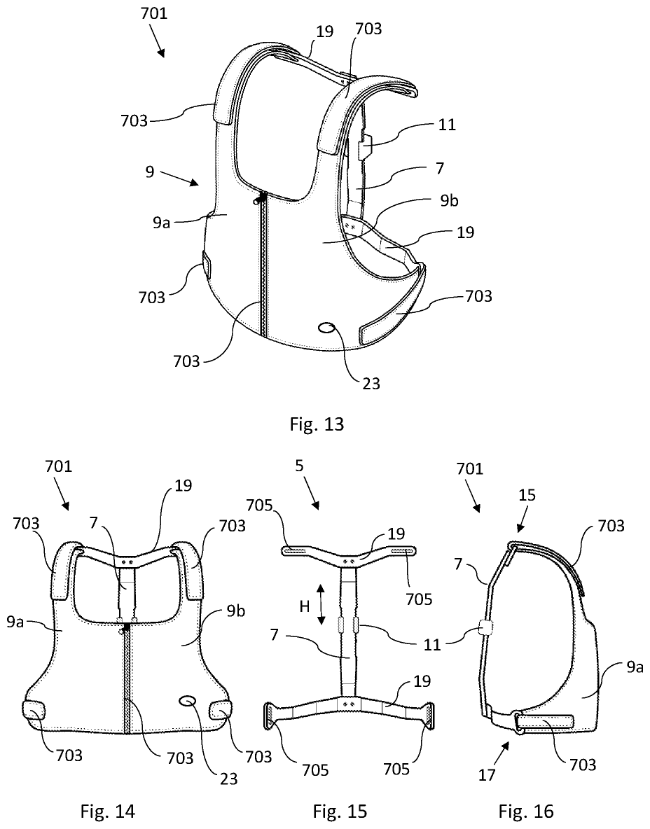

FIG. 13: an isometric view of a torso support according to the present disclosure is shown;

FIG. 14: a front view of the torso support from FIG. 13 is shown;

FIG. 15: an element of the torso support from FIG. 13 is shown;

FIG. 16: a side view of the torso support from FIG. 13 is shown.

FIGS. 1 and 2 illustrate an isometric view of a human body 3 wearing a wearable support system 601.

Wearable support system 601 comprises a torso support 1 for bearing upon a back of a human body 3 and a wearable support structure 13 for supporting the human body 3 during leaning or bending over.

The torso support 1 comprises a first connection arrangement 9, formed as a vest, for connecting the torso support 1 to the human body 3 and a second connection arrangement 11 for connecting the torso support 1 to the wearable support structure 13. The first connection arrangement 9 is connected to the frame 5 at a first location 15 provided at an upper part of the back of the human body 3, and at a second location 17 provided at a lower part of the back of the human body 3. For detecting a physical state of the human body 3 and the deflection of the frame 5, the torso support 1 is provided with a detector arrangement 23.

The wearable support structure 13 is connected via a third connection arrangement 27 thereof to the second connection arrangement 11 of the torso support 1. The wearable support structure 13 comprises two elongated resilient stays 29 connected to the third connection arrangement 27 and extending substantially alongside the human body 3, and two thigh supports 31, each for bearing upon a respective thigh area of the human body 3. Two pivoting arrangements 33, each connecting a respective elongated resilient stay 29 to a respective thigh support 31, allow the respective elongated resilient stay 29 and the respective thigh support 31 to pivot about a main axis A.

The frame 5 of the torso support 1 is connected to a hip part 25 of the wearable support structure 13. The frame 5 comprises a resilient stay 7 extending substantially alongside the back of said human body 3 and a further resilient stay 19 extending along a width of the back of the human body 3, perpendicular to the resilient stay 7. The second connection arrangement 11 is moveable in a direction H along the frame 5 between the first location 15 and the second location 17.

FIG. 3 illustrates a human body 3 wearing a torso support 1 while being in an upright position. While in this position, the resilient stay 7 is in a first state when being unloaded.

FIG. 4 illustrates a human body 3 wearing a torso support 1 while being in a leaning or bending position respectively. In this position, the resilient stay 7 is in a second state. The resilient stay 7 is biased back to the first state when being into the second state, thereby supporting the human body 3 during leaning or bending over.

Furthermore, the two elongated resilient stays 29 as well as the two thigh supports 31 are in the first state when being unloaded corresponding with the upright position of the human body 3, and are biased back to the first state when being into the second state corresponding with the leaning or bending position of the human body 3, thereby supporting the human body 3 during leaning or bending over.

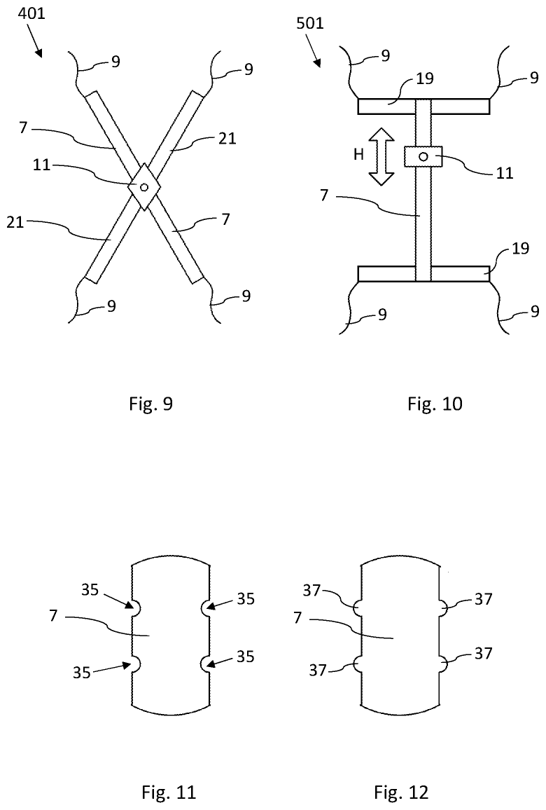

FIGS. 5 to 8 and 10 illustrate further embodiments of a torso support 101, 201, 301, 501 according to the present disclosure comprising connection arrangements 9, 11 and resilient stays 7, 19. FIG. 9 illustrates a yet further embodiment of a torso support 401 according to the present disclosure comprising an X-shaped frame 5 provided with a resilient stay 7 and a further resilient stay 21, wherein the second connection arrangement 11 is provided at the crossing of resilient stay 7 and the further resilient stay 21.

FIGS. 11 and 12 illustrate a detail of the torso support 501 for which the second connection arrangement 11 is moveable in a direction H along the resilient stay 7. Via either protrusions or recesses provided in the second connection arrangement 11 engaging matching recesses 35 or protrusions 37 provided in the resilient stay 7, the second connection arrangement 11 can be fixed to the frame 5 at a plurality of positions between the first location 15 and the second location 17.

Elements of torso support 701 that are similar to elements of torso support 1 and 501 are provided with identical reference numbers. The vest 9 of torso support 701 comprises two vest parts 9a and 9b that are mutually connectable via a connection arrangement 703 such as a zipper or buttons. Each vest part 9a, 9b is provided with straps 705 that may be adhered to a surface of the vest part 9a, 9b for allowing the user to adapt the fitting of the torso support 701 to his or her body for realising a relative tight-fitting for maintaining the resilient stay 7 in the second state during leaning or bending position of human body. To this end, the straps 705 and the vest parts 9a and 9b are provided with hook and loop fasteners (not shown). The further resilient stays 19 are provided with through holes for allowing the straps 705 to be attached to the frame 5.

1. A wearable support system (601) comprising a torso support (1, 101, 201, 301, 401, 501, 701) and a wearable support structure (13), wherein said torso support (1, 101, 201, 301, 401, 501, 701) is arranged for bearing upon a chest and/or a back of a human body (3) and arranged for at least partly supporting said human body (3) during leaning or bending over, said torso support (1) comprising:
a frame (5) comprising a resilient stay (7) arranged to extend substantially alongside said back of said human body (3) and arranged for being in a first state when being unloaded, and arranged to be biased back to said first state when being into a second state corresponding with a leaning or bending position of said human body (3);
a first connection arrangement (9) arranged for connecting said torso support (1, 101, 201, 301, 401, 501, 701) to said human body (3), wherein said first connection arrangement (9) is connected to said frame (5) at a first location (15) that, in use, is provided at an upper part of said back of said human body (3), and a second location (17) that, in use, is provided at a lower part of said back of said human body (3);
a second connection arrangement (11), connected to said frame (5) between said first location (15) and said second location (17), arranged for connecting said torso support (1, 101, 201, 301, 401, 501, 701) to said wearable support structure (13); and
wherein said wearable support structure (13) is arranged for at least further partly supporting said human body (3) during leaning or bending over and arranged for bearing against a lumbar area and/or a thigh area of said human body (3), wherein said wearable support structure (13) comprises:
a third connection arrangement (27) arranged for connecting said wearable support structure (13) to said torso support (1, 101, 201, 301, 401, 501, 701), wherein said wearable support structure (13) is connected to said second connection arrangement (11) via said third connection arrangement (27).

2. The wearable support system (601) according to claim 1, wherein said frame (5) is arranged such that, in use, a shape of said resilient stay (7), between said first location (15) and said second location (17), corresponds to a shape of said back of said human body (3).

3. The wearable support system (601) according to claim 1, wherein said second connection arrangement (11) is rotatably connected to said third connection arrangement (27).

4. The wearable support system (601) according to claim 3, wherein said second connection arrangement (11) is rotatably connected to said third connection arrangement (27) such that said second connection arrangement (11) is rotatable relative to said third connecting arrangement (27) over a predetermined angle for limiting rotation of said torso support (1, 101, 201, 301, 401, 501, 701) relative to said wearable support structure (13).

5. The wearable support system (601) according to claim 1, wherein said third connection arrangement (27) is arranged for applying a force, during use of the wearable support system (601), in a direction away from said human body, by the wearable support structure, to said torso support, via said second connection arrangement.

6. The wearable support system (601) according claim 1, wherein said second connection arrangement (11) is moveable along said frame (5) between said first location (15) and said second location (17).

7. The wearable support system (601) according to claim 6, wherein said torso support (1) is arranged for fixating said second connection arrangement (11) at a plurality of positions between said first location (15) and said second location (17) along said frame (5) to said frame (5).

8. The wearable support system (601) according to claim 1, wherein said frame (5) is detachably connected to said first connection arrangement (9).

9. The wearable support system (601) according to claim 1, wherein said first connection arrangement (9) comprises a textile arranged for bearing upon said chest of said human body (3).

10. The wearable support system (601) according to claim 1, wherein said first connection arrangement (9) is formed as a vest.

11. The wearable support system (601) according to claim 1, wherein said frame (5) comprises a further stay (19), preferably a further resilient stay, perpendicular to said resilient stay (7) such that, in use, said further stay (19) extends along a width of said back of said human body (3).

12. The wearable support system (601) according to claim 1, wherein said frame (5) comprises a yet further stay (21), preferably a yet further resilient stay, wherein said resilient stay (7) and said yet further stay (21) are provided such that said frame (5) is X-shaped, preferably, wherein said second connection arrangement (11) is provided at a crossing of said resilient stay (7) and said yet further stay (21).

13. The wearable support system (601) according to claim 1, wherein said torso support (1, 701) comprises a detector arrangement (23) arranged for detecting a physical state of said human body (3) and/or for detecting forces and deflections of said frame (5).

14. The wearable support system (601) according to claim 1, wherein said frame comprises a neck support for support a neck of said human body.

15. The wearable support system (601) according to claim 1, wherein said frame (5) is arranged for connecting to a hip part (25) of said wearable support structure (13) for limiting rotation of said hip part during donning and doffing of said wearable support structure.

16. The wearable support (601) system according to claim 1, wherein said wearable support structure (13) further comprises:
an elongated resilient stay, preferably two elongated resilient stays (29), connected to said third connection arrangement (27), arranged to extend substantially alongside said human body (3) and arranged for being in a first state when being unloaded, and arranged to be biased back to said first state when being into a second state corresponding with a leaning or bending position of said human body (3).

17. The wearable support system (601) according to claim 1, wherein said wearable support structure (13) comprises:
two thigh supports (31), each arranged for bearing upon a respective thigh area of said human body (3), arranged for being in a first state when being unloaded, and arranged to be biased back to said first state when being into a second state corresponding with a leaning or bending position of said human body (3);
two pivoting arrangements (33), each connecting said elongated resilient stay, preferably each connecting a respective elongated resilient stay (29) of said two elongated resilient stays (29), to a respective thigh support (31) of said two thigh supports (31) and allowing said respective elongated resilient stay (29) and said respective thigh support (31) to pivot about a main axis (A).
